(19) United States
(12) Patent Application Publication
(10) Pub. No.: US 2006/0173439 A1
(43) Pub. Date: Aug. 3, 2006

(54) Syringe drive system

(76) Inventors: Gale H. Thorne JR., Bountiful, UT (US); Gale H. Thorne, Bountiful, UT (US); Michael Wallace Howlett, Salt Lake City, UT (US)

Correspondence Address: Gale H. Thorne, Ph.D., P.A., 1056 Millcrest Circle, Bountiful, UT, 84010, US

(21) Appl. No.: 11/037,900

(22) Filed: Jan. 18, 2005

(51) Int. Cl. A61M 37/00; A61M 31/00

(52) U.S. Cl. 604/506; 604/155

(57) ABSTRACT

A syringe driver or pump system based upon a syringe stem and associated assembly having a spiral threaded pattern along the outer surface of the stem, a collet-button which is displaceable along the stem assembly (as a nut is displaced along a bolt) and a spring. All are disposed to provide a cam action, translating rotary activity of the collet-button, which tends to be displaced to compress the spring when rotated, to linear displacement as the force of energy is released from the spring when compressed. Thus, force available for driving effluent fluid flow from the syringe is limited by the energy available from the spring, rather than force and energy which might be directly applied to otherwise displace the syringe stem assembly and collet-button. A digitally activated syringe driver and an electrical motor activated driver is disclosed. A number of electronic control systems for the motor are also disclosed.

### FIELD OF INVENTION

Inventions which are disclosed herein are related to medical syringes having a barrel and a piston for displacing fluids within the barrel and more specifically to powered systems which are used to drive such syringes to both dispense and draw-up medications and other fluids from and into syringes.

### BACKGROUND AND DESCRIPTION OF RELATED ART

While syringes comprising a barrel and an associated piston have been used in the medical arts for a very long time, use and makeup of syringes are undergoing constant change to keep up with an ever evolving pattern of medical practice.

As examples, needle-bearing syringes are increasingly employing some form of needle guard to protect against dangers of needle sticks. Use of needleless syringes to deliver medications and to flush indwelling catheters is becoming increasingly prevalent and is the standard of practice in many healthcare facilities. Contemporary medications often require timed delivery at controlled rates to assure appropriate medical responses and to guard against vessel trauma and other adverse sequelae resulting from too high concentration or overly fast infusion of a given medicinal drug.

Further, improvements in syringe art have resulted in discovery and manufacture of materials which can reliably store drugs and other related fluids in syringes for long periods of time. Such long term storage of medications and other liquids has precipitated an accelerating growth in commercialized prefilled syringes. Other advancements in syringe art have yielded new prefilled syringe products having multiple chambers from which two or more disparate fluids may be delivered sequentially. One of the major uses of multi-chamber syringes is dispensing of a drug dose through a catheter followed by a flush bolus of an inert liquid to clear the catheter and complete a drug administration cycle by a single stroke of a syringe piston.

Related Art Compendium

Various forms and types of syringe drivers are known and commercially available. A few selected examples of U.S. patents which disclose various types of syringe drivers are as follows:

A spring driven syringe driver is disclosed in U.S. Pat. No. 4,681,566 issued to Paul v. Fenton, Jr., et al. (Fenton) Jul. 21, 1987. Fenton teaches selection of a predetermined spring-generated force to drive a syringe piston.

A syringe drive apparatus comprising a cylindrical barrel with a wall at one end with a nozzle and with a threaded actuating rod extending from the other end is disclosed in U.S. Pat. No. 4,312,343 issued to Harry H. Leveen, et al. (Leveen) Jan. 26, 1982. A collar is affixed to the syringe whereby angular rotation of the rod displaces the rod and an associated piston linearly.

A fluid syringe drive system is disclosed in U.S. Pat. No. 4,744,786, issued to Michael D. Hooven (Hooven) May 17, 1988. A viscous fluid is metered into a proximal end of a syringe to expel a fluid from the syringe at a controlled rate.

U.S. Pat. No. 4,755,172, issued to Brian E. Baldwin Jul. 5, 1988 discloses a syringe driver which applies a frictional driving force directly to a stem of a syringe piston. The drive is powered by a pair of Neg'ator constant force springs.

Use of a threaded rod as a drive member is disclosed in U.S. Pat. No. 4,883,472, issued to Peter Michel (Michel 472) Nov. 28, 1989. Adjustment of a manipulating head permits preselection of an arbitrary amount of liquid to be injected by pressure placed upon the manipulating head. An earlier U.S. Pat. No. 4,585,439, issued to Peter Michel (Michel 439) Apr. 29, 1986 also discloses use of a threaded piston rod. The piston rod is driven by a driver sleeve to directly advance a piston of an associated syringe.

U.S. Pat. No. 4,931,041, issued to Ulrich Faeser (Faeser) Jun. 5, 1990 discloses an infusion syringe pump which utilizes a motor-gear to accomplish a linear drive. A position-defining element is connected only to the linearly movable drive member which actuates a syringe piston.

An example of a syringe with a threaded stem is found in U.S. Pat. No. 5,507,727 issued to Lawrence Crainich (Crainich). The piston of Crainich is a threaded rod engaged by a threaded member, the rod being advanced by rotation of a proximally affixed knob. The threaded member is used to thrust the rod forward to expel fluid from an associated syrnge.

U.S. Pat. No. 5,954,695, issued to Nathaniel M. Sims, et al. (Sims) Sep. 21, 1999 discloses a multi-dose syringe driver which effects controlled parental infusion of a medical fluid. Flow rate from the associated syringe is determined by diameter of an attached mircrobore tubing.

A microcontroller controlled infusion device is disclosed in U.S. Pat. No. 6,723,072 B2, issued to J. Christopher Flaherty, et al. (Flaherty) Apr. 20, 2004. The dispensing of fluid using the Flaherty device results from successively applying a charge and removing the charge from a shape charge element.

Additional Background

As it is currently common practice to medicate patients using syringes to dispense liquids through catheters, powered syringe drivers are being used in ever greater numbers. These syringe drivers provide hands-off operation, permitting medication to be dispensed while a clinician is attending to other duties. However, while syringe drivers are used for such purposes in large numbers in U.S. Hospitals, cost of most such drivers often precludes wider use. Specialized use of syringe drivers in hospitals has resulted in ever increasing sophistication of these devices. In addition, a number of syringe driving systems have been recently incorporated into many standard pole-mounted IV pumps to accommodate such needs.

Use of automatically operating powered syringe drivers and pumps has resulted in the introduction of drivers and pumps which provide programmable fluid delivery rates, detection and alarms for over-pressure, anti-free flow features, dose completion signals and programmable drug data bases with automatic lock-out and other features which provide automatic alerts and alarms against improper delivery of medications. It is duly noted that it is not sufficient to generate an alarm on an over-pressure condition; there should be an inherent feature of a syringe driver which assures no over-pressure condition can exist during operation.

Of major concern is the need to detect fluid path blockage so timely corrective action can be taken. It is important to be responsive to patient discomfort or pain by adjusting flow rate when possible. As a result, flow rate control and alarm functions are well known in contemporary syringe drivers. In some cases, it is just as important, in manual syringe operation, to be able to limit a dispensing rate to meet rate-of-delivery specifications and other safety parameters associated with a given drug delivery.

Type of drug to be delivered and area of delivery also play a part in determining requirements and features of syringe delivery systems. For example, some drugs (e.g. gentamicin) must be infused over a specific period of time. Coordinated laboratory tests may be performed to test peaks and troughs in blood serum concentration to evaluate efficacy of the prescribed treatment. For this reason, a full drug dose must be delivered and a catheter flushed in a predetermined time frame. It is common practice for all drugs to be flushed-in with an inert liquid such as saline after introduction of the drug into a catheter or IV line.

Also it may be desirable to deliver sequential doses from a multi-chamber syringe at variable rates. For example, drug delivery may be at a first rate, catheter flush may be at a second rate and a catheter keep open flow (to avoid reflux complications) may be delivered at a third rate.

There are also special requirements for syringe drivers employed in home care. Of paramount importance is simplicity and facility of use for a user patient, particularly the very elderly and weakened. Rate-sensitive infusions must be inherently controlled by a syringe driver or other IV pump in such situations to guard against undesirable side affects of drug infusion, such as vessel irritation (phlebitis).

Nursing home care is a very cost conscious environment where IV therapy is a common, but not consistent treatment modality. In such cases, syringe drivers or pumps may be capital intensive, but still are desirable in a work environment which is personnel limited.

It may be noted that syringe drivers, used with syringes, are known to be able to be provided at a lower cost and also provide a more mobile alternative when compared to other types of parenteral fluid pumps in current use. These other types of pumps are generally used to deliver medications, usually antibiotics from partial-fill bags which can cost ten to fifteen times more than an empty syringe. One of the limitations of use of syringe drivers is a lack of an inherent flushing system. Some of the other pumps have built in flushing systems (e.g. piggyback systems) which automatically flush after delivery of a medication.

### BRIEF SUMMARY AND OBJECTS OF THE INVENTION

In brief summary, this novel invention alleviates all of the known problems related to providing a wide range-of-application syringe driver system. The syringe driver system is primarily used to dispense fluids from a medical syringe having a partially closed distal end through which fluid is dispensed, an open proximal end and a cylindrical barrel therebetween, the barrel preferably having a pair of gripping extensions which extend laterally and radially outward at the proximal end.

Basic to the driver system is an elongated piston or stem of the medical syringe which is securely affixed at one end to a stopper or plunger which occludes and is linearly displaced to propel fluid within the cylindrical barrel of the syringe. Proximally disposed from the stopper or plunger is a grooved stem section, the grooves of which are spirally oriented to form a screw pattern having a predetermined pitch.

Preferably, securely, but releasibly affixed on the other end of the piston is a disk-shaped collet-button which may be used to grip and displace the piston, while there, or broken free to provide a rotational, interface for displacing the stem and piston as an inherent part of a syringe driver system. The collet-button may be so affixed to the stem by a heat stake. The collet-button generally has a hollow core with internal, nut-like spiral threads which are sized and shaped to correspond to the screw pattern of the stem such that the collet-button may be facilely rotated to be displaced along the stem. Further, the collet-button has proximally disposed surface features which provide a quick-connect interface to a drive part of a syringe driver. The collet-button may also have a knurled outer rim which provides a manual gripping surface and a ratchet interface, the purposes of which are fully disclosed hereafter.

Of singular importance is a collet driver associated with the piston. Generally, the collet driver is disposed within a driver housing which is securely affixable to lateral extensions or gripping wings of the barrel. A motor is disposed within the housing in line with the barrel when affixed to the housing. The motor should have sufficient torque, when communicated through the collet driver, to displace the piston to dispense fluid from the syringe.

The collet driver includes a drive shaft or linkage which is directly connected to the motor and a driver part which is angularly displaced by the shaft but upon which the driving part is free to linearly slide. The driver part has distally disposed features which provide complimentary connections for the quick connect interface to the collet-button.

Of primary importance is an energy storage device disposed in line with the drive shaft or linkage between the driver part and motor. Displacement of the driver part toward the motor stores energy in the energy storage device. Release of energy from the energy storage device linearly forces displacement of the driver part against the collet-button, which is coupled to the stem through the threads and grooves, to propel the piston plunger to dispense fluid from the syringe. It is notable that pressure which results from energy released from the energy storage device is limited by energy stored therein and, therefore, may be thereby limited to not exceed a predetermined value independent of torque being produced by the motor. Thus, activating the motor to rotate the driver part to displace the collet-button along the stem in a direction toward the motor stores energy in the energy storage device and ultimately results in a force-limited displacement of the piston to dispense fluid from the syringe. Note that the change of motion from rotary action of the motor, driver part and collet-button to linear displacement of the stem is a cam interface. The energy storage device is preferably a spring.

Preferably, the motor is intermittently driven in an “on” and “off” cyclic fashion. To provide a predetermined flow rate, the motor is turned “on” for a predetermined period of time (to rotate the drive part and associated collet-button through a predetermined angle) relative to another predetermined period for the “off” time. The amount of fluid dispensed is a function of linear displacement of the collet-button which is dependent upon the pitch of stem grooves and corresponding collet-button threads. For this reason, neither the stem nor associated stop or plunger should rotate while the collet button is being driven.

When the motor is “on”, the collet-button is displaced to store energy into the energy storage device (e.g. a spring), although the energy storage device may be simultaneously linearly displacing the piston to dispense fluid from the syringe. When the motor is “off”, the energy storage device continues to release any stored energy by proceeding to displace the piston to dispense additional fluid from the syringe.

In those cases where effluent from the syringe does not permit the complete release of stored energy during the intermittent drive cycles, more and more energy is stored in the energy storage device and the collet-button and drive part are displaced ever closer toward the motor. Such a condition may occur when the drive's system fluid dispensing rate is lower than the motor drive rate, such as when an occlusion is reducing outflow or when the syringe is empty. In such cases, it is expedient to sense such a condition, respondingly remove power from the motor and provide an alert. For this purpose, a sensor is disposed to sense a limit point of such displacement. It is preferred that power be removed from the motor drive when such a displacement condition is sensed.

A syringe driver according to the instant invention may be provided in a variety of models ranging from a simple variable rate syringe driver to a device which can manage drug infusion, providing such features as programmable drug data bases with automatic lock-out, alerts and alarms. For these purposes a bar code reader and microprocessor may be added to provide an electronic control system.

In simplest format, a syringe driver may not employ a motor or other mechanical energy producing device and may be operated manually. In some medical delivery applications, it s preferable to deliver by syringe, but at a rate which is slower than that conveniently achievable by manually depressing a stem of a syringe. For this purpose, a snap-on apparatus may be employed to constrain the delivery rate. The snap-on apparatus is affixed to the syringe and disposed about a collet-button to deter directly pushing the stem into the syringe barrel to dispense fluid.

The snap-on apparatus has lateral openings which provide access to the outer rim of the collet-button whereby the collet-button may be manually articulated to drive the stem linearly and generally at a slower rate than that of a directly pushed stem. However, in the case of an apparatus which is so driven, just as in the case of a motor driven device, over-pressure situations must be prevented. Also, it is desirable to be able to retract the stem a short distance, such as the distance to draw in a desired amount of fluid into the syringe to test for blood flash.

To satisfy both of these conditions a spring is disposed in the snap-on apparatus proximally disposed relation relative to the collet-button. A pawl is provided to interface with the ratchet pattern of the outer rim of the collet-button to limit articulation of the collet-button to a direction of rotation which stores energy into the spring rather than to drive the stem to directly dispense fluid from the syringe. Note that the stem of the syringe may be retracted a short distance (compressing the spring) to test for blood flash while articulation of the collet-button simply stores energy in the spring which reactively displaces the stem to dispense fluid from the syringe with forces restricted to the force which may be stored in the spring. Note that, once a spring is fully compressed, no additional force may be applied to the stem by rotating the collet-button.

Method for use of either the syringe driver or snap-on apparatus is simple. Either the syringe driver or snap-on apparatus is disposed about a collet-button and affixed to the lateral extensions of the barrel (such as by a bayonet attachment to syringe gripping extensions or flanges).

In the case of the syringe driver, the rate at which fluid is to be dispensed is selected and power is turned “on” to the motor. Powered infusion continues at the selected dispensing rate until manually stopped, a flow alert is sensed or the associated syringe is emptied. Note that by nature of the stored energy device, reflux does not occur when power is removed from the motor (due to force of energy stored in the spring).

In the manual system, fluid dispensing rate is similarly controlled by energy stored in the energy storage device (e.g. a spring). Such a spring is powered by articulation of the collet-button. At each point where articulation ceases, reflux is prevented by pressure exerted by the spring.

Further, the syringe driver may be used to dispense disparate fluids from multi-chamber syringes. In such cases, it may be desirable to dispense fluids from the separate chambers at different rates. In such cases, a sensor may be used to determine varying patterns of displacement of the driver part against the energy storage device by programming within the microprocessor. Pattern recognition programs may be used to detect such events as by sensing a valve opening or change or resistance when the plunger is displaced to provide decision milestones at which flow rates are varied.

Accordingly, it is a primary object to provide a syringe driver system which is driven by a high torque motor, but which cannot over-pressure a syringe and associated attachments.

It is another primary object to provide a piston or stem of a syringe which comprises a plurality of grooves along the piston or stem which are spirally oriented to form a screw pattern having a predetermined pitch for use in a cam interface used to transfer rotational displacement of a motor to linear displacement of the piston or stem.

It is a consequential object to provide a collet-button which is releasibly affixed to a proximal end of the piston or stem for gripping purposes and which may be released from attachment to the proximal end of the piston or stem to be rotationally displaced along the piston or stem for use in the cam interface.

It is an important object to provide the collet-button with a proximally facing structure whereby a driver part connects thereto as part of the cam interface.

It is an object to provide a syringe driver having a motor aligned with a piston or stem of a syringe.

It is a very important object to provide an energy storage device into which energy is stored through the cam interface and which responsively linearly displaces the piston or stem of a syringe to dispense fluid therefrom.

It is an important object to provide circuit control for a motor which controls operational rate of such motor to further control a fluid dispensing rate of an associated syringe thereby.

It is an object to provide a syringe driver with a manually selectable dispensing rate.

It is another very important object to provide sensors and alerts for conditions of excessive flow resistance and an emptied syringe.

It is an object to provide a housing for the syringe driver which has facile and releasible attachment apparatus for attaching the driver to a syringe.

It is an object to provide an electronic control system for a syringe driver which comprises a microprocessor.

It is an object to provide an electronic control system for a syringe driver which comprises a bar code reader.

It is a key object to provide a cam interface between the motor and the syringe piston or stem, said interface being disposed to transform rotational displacement to a linear displacement of a collet-button displacement, which displacement is opposite to the direction of the piston or stem when dispensing fluid from the syringe.

It is an object to provide a method for determining a syringe driver state which exhibits high flow resistance and syringe empty and produces alerts therefore.

It is an important object to provide a syringe driver which inhibits reflux of fluid proximally toward said syringe when power is removed from said motor.

It is another key object to provide a collet from the gripping part of a syringe piston or stem and which rotates thereupon as a nut rotates upon a screw.

It is a basic object to provide an electronic control system which intermittently drives a driver part with a torque from a motor which would yield an over-pressure force to a piston if driven directly to a plunger piston, but which provides a reduced and smoothed reasonably acceptable pressure to the piston through the energy storage device by actuating the driver part to intermittently drive against the energy storage device and therethrough to the plunger piston.

It is yet another primary object to provide a snap-on lock apparatus whereby a collet-button, when disposed at the proximal end of the barrel, is securely affixed by the apparatus to inhibit linear displacement of the collet-button while permitting rotation thereof to propel the piston or stem linearly.

It is an object to provide the lock apparatus with at least one direction retarding pawl and a collet-button with corresponding ratchet teeth to thereby restrict collet-button rotation to a single direction such that collet-button rotation displaces the collet-button away from said syringe barrel.

It is an object to provide a lock apparatus with a spring housed in a compartment, the spring storing energy from collet-button rotation and acting to force dispensing of fluid from an associated syringe.

It is an object to provide a manual syringe drive apparatus and associated method of use which assures manually applied torque does not directly drive fluid from the syringe.

It is an object to provide a syringe driver and associated method of use which assures motor torque is not directly applied to a piston or stem of a syringe to drive fluid from the syringe.

These and other objects and features of the present invention will be apparent from the detailed description taken with reference to accompanying drawings.

### BRIEF DESCRIPTION OF THE DRAWINGS

FIG. 1 is a perspective of an exemplary commercial syringe with a piston and stopper assembly disposed within the barrel of the syringe (prior art).

FIG. 1A is a section of the syringe seen in FIG. 1 taken along lines 1A-1A (prior art).

FIG. 2 is a section of a syringe, similar to the section seen in FIG. 1A, but with a valve assembly distally disposed relative to a piston and stopper similar to the piston and stopper of the syringe of FIG. 1.

FIG. 2A is a magnified portion, taken along lines 2A-2A, of the syringe seen in FIG. 2.

FIG. 3 is a perspective of a syringe made according to the invention.

FIG. 3A is a schematic cross-section, taken along lines 3A-3A, of the syringe seen in FIG. 3.

FIG. 4 is a perspective of a piston or stem assembly of the syringe seen in FIG. 3.

FIG. 5 is a perspective of the collet-button seen in FIGS. 3 and 4.

FIG. 6 is a perspective of the syringe seen in FIG. 3 with a collet-button portion of the piston or stem assembly displaced distally toward the barrel of the syringe.

FIG. 6A is a schematic cross-section, taken along lines 6A-6A, of the perspective seen in FIG. 5.

FIG. 7 is a perspective of the piston or stem of the syringe assembly seen in FIG. 4.

FIG. 8 is a perspective of a piston or stem assembly similar to the piston or stem assembly seen in FIG. 7.

FIG. 9 is a perspective of a syringe driver assembly affixed to the syringe seen in FIG. 3.

FIG. 10 is a perspective of a section taken along lines 10-10 of FIG. 9 and rotated for a better view of interconnecting linkage between the driver assembly and the syringe.

FIG. 11 is a perspective of parts of the driver seen in FIG. 9, with a housing removed, and associated syringe.

FIG. 12 is another perspective similar to that of FIG. 11 with additional parts removed and with a collet-button displaced to abut the barrel of the associated syringe.

FIG. 12A is a schematic cross-section, taken along lines 12A-12A, of the perspective seen in FIG. 12.

FIG. 13 is a schematic cross-section of the driver, similar to the cross section seen in FIG. 12A, seen unattached to a syringe.

FIG. 14 is a perspective of a syringe driver drive linkage with an associated spring extended when unattached to a syringe as seen n FIG. 13.

FIG. 15 is a perspective of a drive cylinder and drive stem associated with the driver seen in FIG. 9.

FIG. 16 is a perspective of a syringe and driver assembly similar to the perspective of FIG. 12, but with drive cylinder, drive stem and collet-button rotated along the associated piston or stem away from being abutted against the barrel of the associated syringe.

FIG. 16A is a schematic cross-section, taken along lines 16A-16A, of the perspective seen in FIG. 16.

FIG. 17 is a perspective of a syringe and driver assembly similar to the perspective of FIG. 16, but with the drive cylinder, drive stem and collet button rotated and displaced such that the drive cylinder is in contact with a limit sensor.

FIG. 17A is a schematic cross-section, taken along lines 17A-17A, of the perspective seen in FIG. 17.

FIG. 18 is an example of a simplified control circuit for a driver made according to the invention.

FIG. 19 is another example of a simplified control circuit of a driver made according to the invention.

FIG. 20 is a digital control circuit schematic for a driver made according to the invention.

FIG. 21 is a digital control circuit schematic similar to the schematic seen in FIG. 20, but showing uses of a microprocessor to perform driver control functions.

FIG. 22 is a motor drive timing chart showing motor drive “on” and “off” periods.

FIG. 23 is a motor drive timing chart showing motor drive “on” and “off” periods, similar to FIG. 22, but with longer “on” periods.

FIG. 24 is a motor drive timing chart showing motor drive “on” and “off” periods, similar to FIG. 23, but with longer “off” periods.

FIG. 25 is a spring displacement timing chart showing a response to the “on” and “off” motor drive periods of FIG. 24.

FIG. 26 is a spring displacement timing chart, like the chart of FIG. 25, but showing different spring displacement.

FIG. 27 is a motor drive timing chart similar to FIG. 24, but showing cessation of motor drive upon displacement of spring reaching a predetermined threshold.

FIG. 28 is a spring displacement timing chart similar to FIG. 26 showing another spring displacement pattern.

FIG. 29 is a motor drive timing chart similar to FIG. 24, but showing cessation of motor drive upon displacement of spring reaching a predetermined threshold.

FIG. 30 is a program flow diagram for a microprocessor based driver control system.

FIG. 31 is a perspective of a snap-on lock apparatus affixed to a syringe whereby an associated stem (not shown) may be manually displaced.

### DETAILED DESCRIPTION OF THE ILLUSTRATED EMBODIMENTS

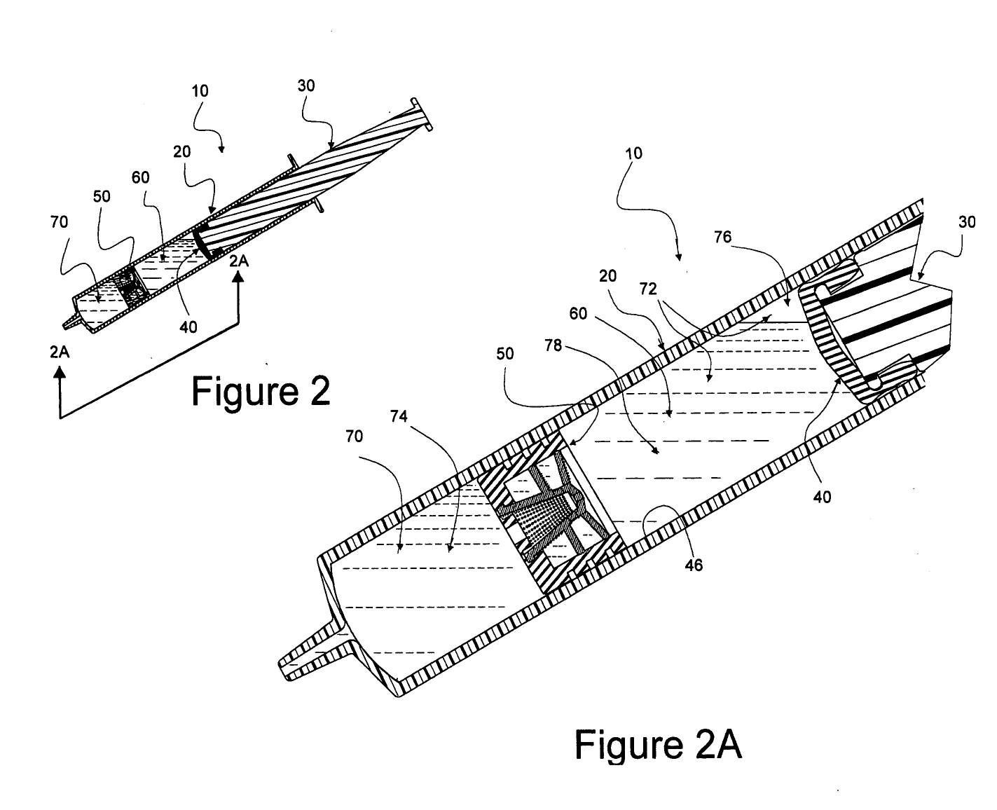

This invention is supportive of both single chamber syringes and multi-chamber syringes such as those disclosed in U.S. patent application Ser. No. 10/838,101, titled MULTI-CHAMBER, SEQUENTIAL DOSE DISPENSING SYRINGE and filed May 3, 2004 by Howlett, et al (Howlett '101). Multi-chamber syringe parts seen in FIGS. 2 and 2A are disclosed in detail in Howlett '101 and are included by reference herein.

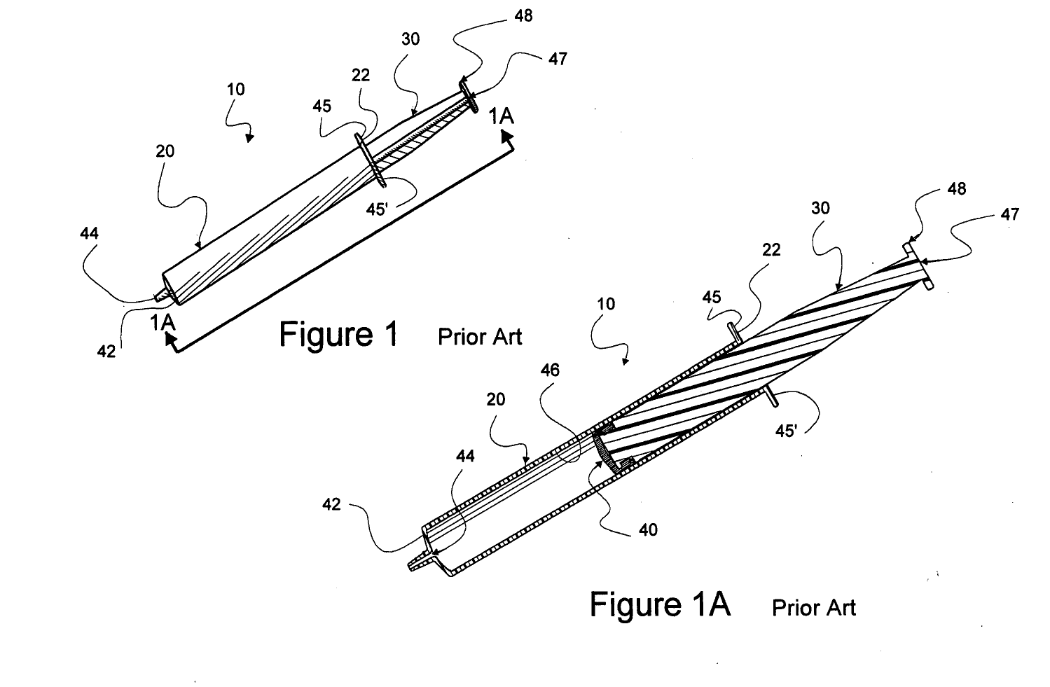

In this description, the term proximal is used to indicate a portion of a device normally closer to a clinician using the device or, in other words away from a patient. The term distal refers to an oppositely disposed portion. Reference is now made to embodiments illustrated in FIGS. 1-31 wherein like numerals are used to designate like parts throughout. Primes of numbers are used to represent parts which are similar, but not identical to other parts having the same numbers.

As used herein, the term “fluid” is defined to be a substance (either liquid or gas) which tends to flow or to take the shape of its container. The term “gas” is defined to be a fluid that expands indefinitely and which may be understood in most circumstances within the scope of this document to be consistent with air. The term “liquid” is a fluid which is free flowing like water, but which is neither solid nor gaseous. Liquids, like water, disclosed in this disclosure are generally understood to be incompressible.

Prior art syringes (as exemplified by syringe 10 in FIGS. 1 and 1A), are available from a large number of commercial companies worldwide. Such syringes typically comprise an elongated hollow syringe barrel, generally numbered 20, which is open at a proximal end 22 to receive a syringe piston (specifically numbered 30 in this embodiment), and a stopper 40 and closed at a distal end 42 about a fluid transmission orifice 44. Generally, barrel 20 is of substantially constant diameter (within tolerances allowed by manufacturing methods, such as by injection molding for barrels made from synthetic resinous materials). Also, generally, barrel 20 has a pair of proximally disposed, laterally extending gripping members 45 and 45′. Stopper 40 is compressible and sufficiently elastic when compressed to provide an efficient wiping action along the length of an internal cylindrical surface 46 of barrel 20. At a proximal end 47, piston 30 has a planar, disk shaped button 48 which facilitates handling and linear displacement of piston 30 within barrel 20.

As seen in FIG. 2 and disclosed in more detail in Howlett '101, a valve assembly 50 is inserted into barrel 20 to divide space within barrel 20 into a proximal chamber 60 and a distal chamber 70. As seen in FIGS. 2 and 2A, each chamber, 60 and 70, may be filled with a bolus of fluid, 72 and 74, respectively. It may be noted that, when chamber 60 is substantially filled with a bolus of fluid (which should be mostly an incompressible liquid), displacement of stopper 40 results in substantially the same displacement of valve assembly 50. Solutions for problems related to a small quantity 76 of gas (e.g. air) trapped in chamber 60 are provided in Howlett '101).

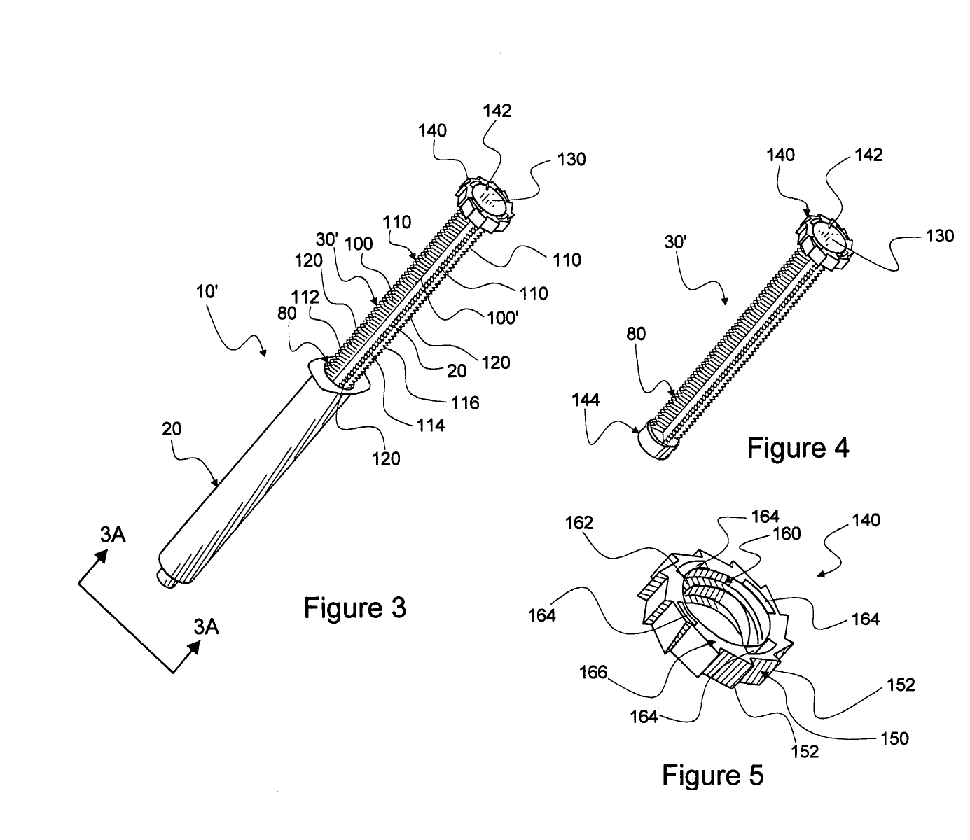

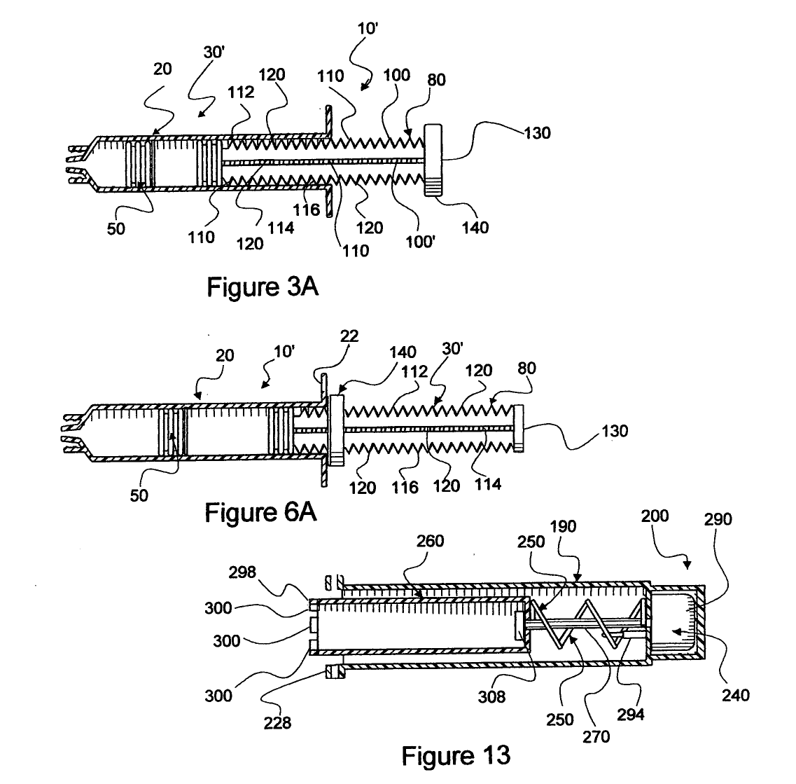

Reference is now made to FIGS. 3 and 3A wherein a syringe 10′, made according to the instant invention, is seen. Syringe 10′ has a standard barrel 20 and a syringe piston or stem assembly 30′. As is the case of syringe piston 30 (see FIG. 1), the stem portion 80 of assembly 30′ is made from two orthogonally intersecting planes (numbered 100 and 100′). However, each outer edge, generally numbered 110, of each plane 100 and 100′ comprises a pattern of teeth (generally numbered 120). Note that only three edges 110 are seen in FIGS. 3 and 3A, and are individually numbered 112, 114 and 116. The number 118 is reserved for a fourth edge, if shown, but hidden in FIGS. 3 and 3A.

Teeth 120 on edges 112, 114, 116 and 118 are organized in a spiral pattern, much like threads on a screw, which has a predetermined pitch and spacing, the purpose of which is fully disclosed hereafter. Also, the general geometric construction of teeth pattern 120 permits a threaded member to facilely be rotated about edges 110.

On a proximal end 130 of assembly 30′, a collet-button 140 is securely, but releasibly affixed thereto. While collet-button 140 may be affixed to stem 80 mechanically or with adhesive, in this case collet-button 140 is thereat affixed by a heat stake 142 (see FIG. 3). Assembly 30′ is better seen without syringe 10′ in FIG. 4. Securely affixed at a distal end of Assembly 30′ is a stopper or plunger 144. Stopper 144 is sized and shaped to be compressed when disposed within barrel 20 to completely expel fluid from distal end 44 (see FIG. 3) of barrel 20 when distally displaced. It may be noted that in each FIG. 3A, 6A, 12A, 16A and 17A an optional valve assembly 50 is disposed in barrel 20. These are provided to emphasize opportunity to use the syringe driver of the instant invention in multi-chamber syringes.

As seen in FIG. 5, collet-button 140 has a nut or nut-like shape. Though not necessary for a motor driven syringe driver, collet-button 140 has an outer circumference 150 comprising a series of notches, generally numbered 152, which may be used as ratchets or as a knurled surface to manually articulate collet-button 140 about and, therefore, along stem 80. For this purpose, the hollow inner cylindrical core 160 of collet-button 140 comprises a spiral thread pattern 162. The pitch and geometry of pattern 162 is sized and shaped to permit collet-button 140 to be facilely articulated along stem 80. In addition, collet-button 140 has a series of slots, generally numbered 164, disposed about the proximal face 166, thereof. Purpose and function of slots 164 are disclose in detail hereafter.

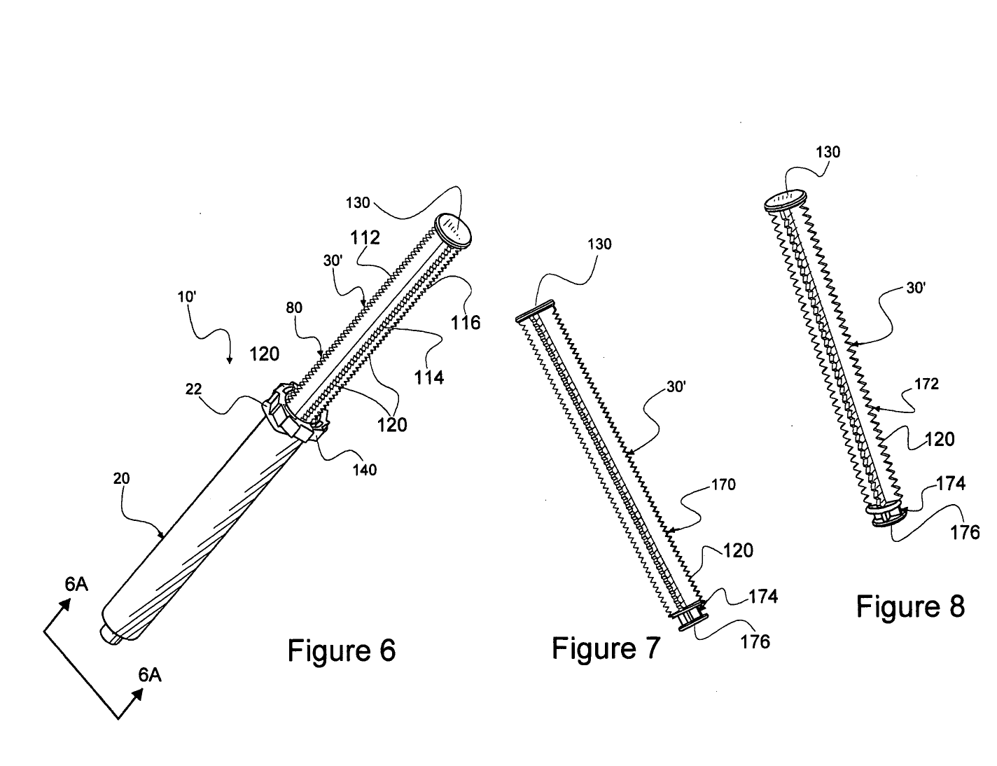

As seen in FIGS. 6 and 6A, collet-button 140 has been frangibly released from end 130 of assembly 30′ and rotated distally to be displaced against proximal end 22 of syringe barrel 20. Once so displaced, collet-button 140 (as well as syringe 10′) is ready for attachment of a driver made according to the instant invention. It should be noted that rotating collet-button to be displaced proximally away from end 22 provides a gap whereby assembly 30′ may be linearly distally displaced relative to barrel 20. The distance of such displacement (rotational displacement of collet-button 140 about stem 80) is dependent upon the pitch of the spiral pattern of teeth 120 on edges 112, 114, 116 and 118. The amount of fluid which may be dispensed by such a displacement is dependent upon the length of the displacement times the area defined by the inner circumference of barrel 20.

And so, as seen in FIGS. 7 and 8, patterns of teeth 120 may be varied to provide different rates of effluent flow from syringe 10′. Note that pattern 170 of teeth 120 (on assembly 30′) seen in FIG. 7 compared to pattern 172 of teeth 120 (on assembly 30″) in FIG. 8 requires a larger angular rotation of collet-button 140 to expel the same volume of fluid from syringe 10′. There are many factors which determine a desired pitch on such patterns. The factors include, but are not limited to, precision of liquid to be dispensed, mechanical gain desired for motor action and limiting friction and stiction effects in piston and plunger displacement.

Note connecting geometry 174 of each distal end 176 of stem 30′ and 30″. Similar connecting geometry is commonly found for securely connecting a stopper (e.g. stopper 144, see FIG. 4) to a stem (e.g. stems 30′ or 30″). However, care must be taken in sizing and shaping both stems and stoppers as neither should rotate as collet-button 140 rotates about an associated stem. Such rotation would reduce effectiveness and flow control accuracy of a driver made according to the invention which rotates collet-button 140 about the associated stem as part of a cam system which ultimately forces fluid from syringe 10′.

Further, material from which collet-button 140 and the associated stem (e.g. 30′ or 30″) is made should be sufficiently sturdy to stand-up under stress of torque of a drive motor and should be sufficiently self-lubricating to reduce lateral forces, due to friction, to a value which does not overcome stiction of the combination of stopper 144 and the associated stem. Polypropylene may be used for material for both a collet-button and a stem.

Also, when selecting a pitch for a given pattern 172 of teeth 120, consideration should be given to the amount of lateral force which results from a selected pitch. Even though a higher pitch angle provides opportunity for greater volumetric effluent flow per unit angle of rotation of collet-button 140, it may be advisable to select a reduced angle to assure a lower, more acceptable lateral force which results from forcing collet-button 140 to rotate.

Another factor for consideration is use of a single driver with a single volumetric calibration for dispensing known volumetric delivery rates of liquid from syringes of different sizes. As an example, if a first syringe had an inner barrel diameter of “d₁” (with a stem assembly 30′ and pattern 170) and a second syringe (not shown) had an inner barrel diameter of “d₂” (with a stem assembly 30″ and pattern 172), a ratio of pitch of pattern 170 relative to pitch of pattern 172 would be d₂²/d₁² to yield the same effluent flow rate for the same angular rotation of collet-button 140.

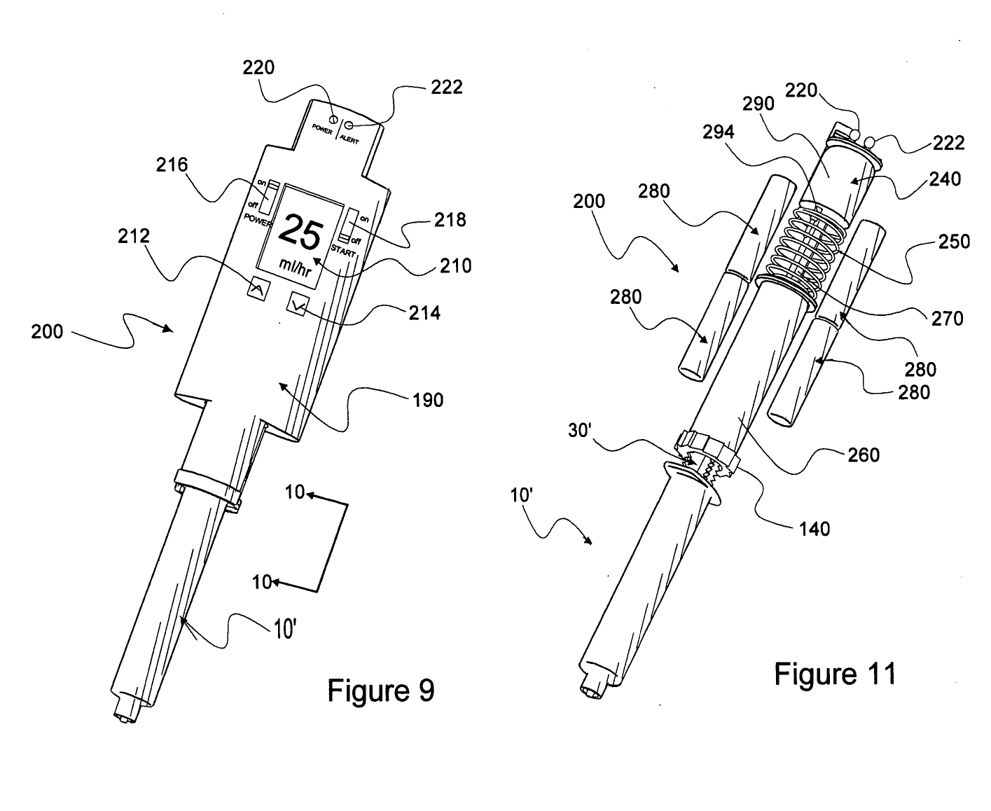

Reference is now made to FIG. 9 wherein an exemplary driver housing 190 for a driver 200 made according to the instant invention is affixed to a syringe 10′. Driver 200 comprises a flow rate indicator 210, two buttons (numbered 212 and 214) for changing driver 200 flow rate, a power control button 216 and driver procedure start button 218. In addition, a power “on” indicator light 220 and an alert light 222 provide illuminated driver 200 status signals.

Housing 190 is designed to contain active parts of driver 200 and to protect a user from moving parts. Housing 190 is preferably injection molded from a high impact plastic such as an acrylic. Further housing 190 is also preferably molded in two parts which are securely affixed for normal use, but which may be opened for access to batteries. Such housing design is well known in the housing design and molding arts.

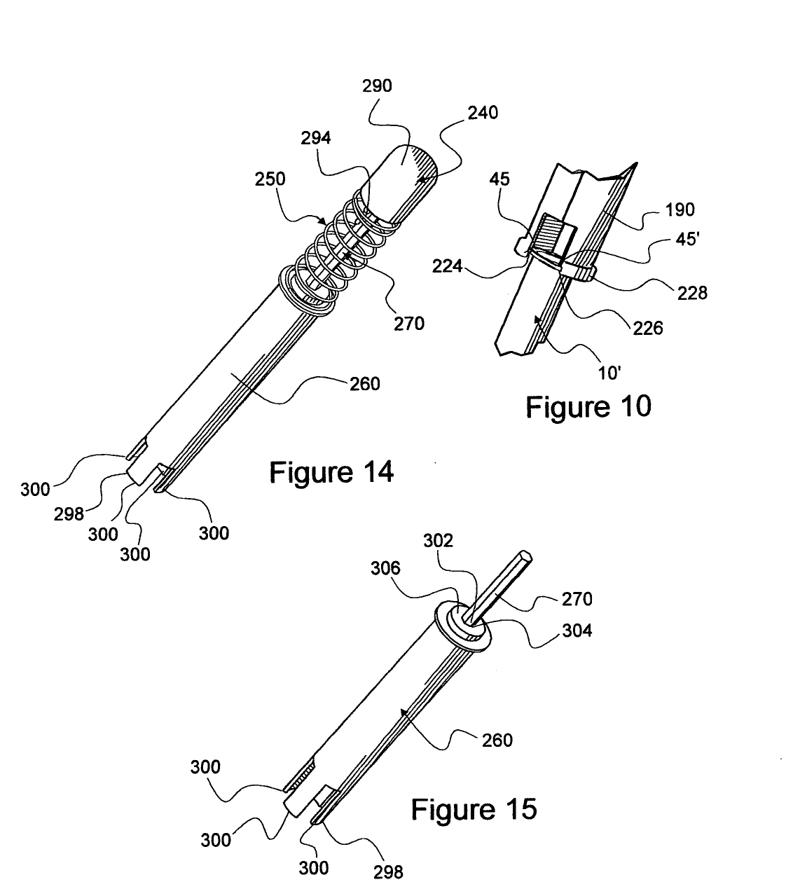

As seen in FIG. 9 and better seen in FIG. 10, housing 190 (and driver 200) is affixed to syringe 10′ via a bayonet type connection whereby a pair of arcing grooves 224 and 226 in a distal portion 228 of housing 190. Arcing grooves 224 and 226 are articulated about outwardly protruding, lateral extensions or flanges 45 and 45′, respectively, of syringe barrel 20, permitting a quarter turn attachment. AS cited supra, extensions or flanges, like extensions 45 and 45′, are generally found on medical syringes.

A complement of parts used in driver 200 is seen in FIG. 11. However, most electronics and associated wiring for driver 200 are not shown in FIG. 11 to clarify presentation of mechanical parts. Schematic diagrams of electrical and electronic control systems are provided hereafter.

As seen in FIG. 11, driver 200 comprises a motor assembly 240, an energy storage device (spring 250), a driver part or drive cylinder 260, a drive shaft 270, a set of batteries, generally numbered 280. Motor assembly 240 comprises a motor 290 and a sensor 294.

Motor 290 is preferably a relatively high torque motor, such as a motor used in a hand held screw driver. It should have sufficient torque that pulsing of the motor for a predetermined period of time causes the motor to rotate an associated motor drive through a predetermined arc. For this reason, a stepper motor may be preferred. Such motors are contemporarily available commercially.

Spring 250 is a compression spring which, when compressed by attachment of driver 200 to a syringe 10′, yields a spring force of sufficient strength to overcome stiction of an associated stem or piston assembly 30′ when collet-button 140 is disposed as seen in FIG. 6A. Further, spring 250 should have a spring constant which limits force exerted by spring 250 to a desired, predetermined force when spring 250 is fully compressed as seen in FIGS. 17 and 17A. Generally, for example, it may be preferred that, for properly lubricated plungers, the range of forces exerted upon collet-button 140 range vary approximately two pounds or greater to not greater than fourteen pounds for a 20 ml syringe.

Reference is now made to FIG. 13 wherein a distal end segment 298 of cylinder 260 extends outwardly from distal portion 228 of housing 190. As seen in FIG. 13, spring 250 is uncompressed. At end segment 298, cylinder 260 comprises a plurality of arcuate fingers, generally numbered 300. Fingers 300 are sized and shaped to fit within slots 164 (see FIG. 5) to rotate collet-button 140 as cylinder 260 is rotated.

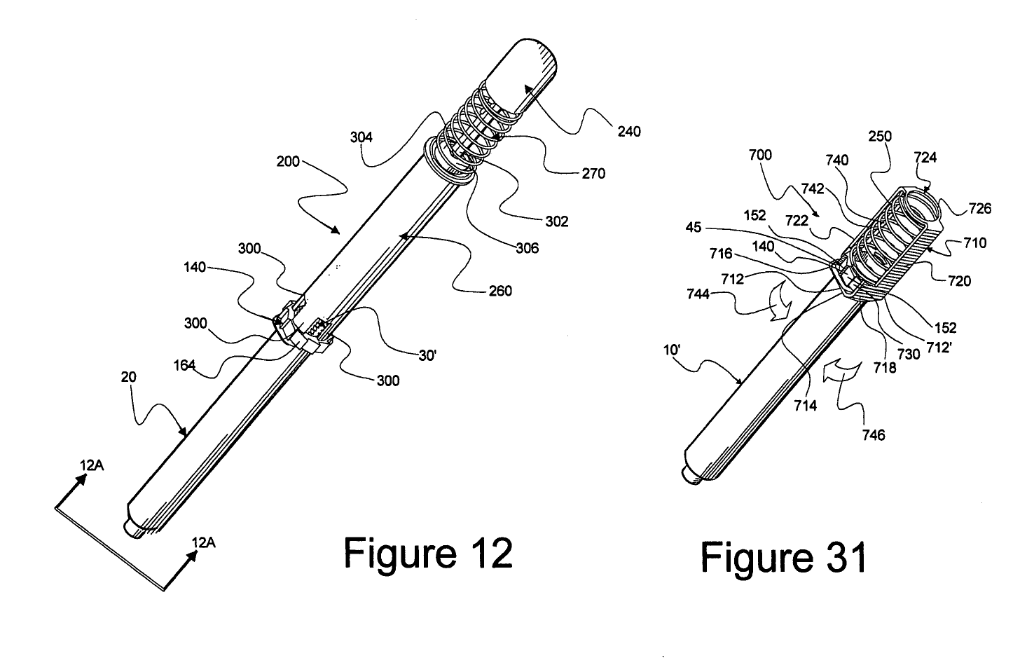

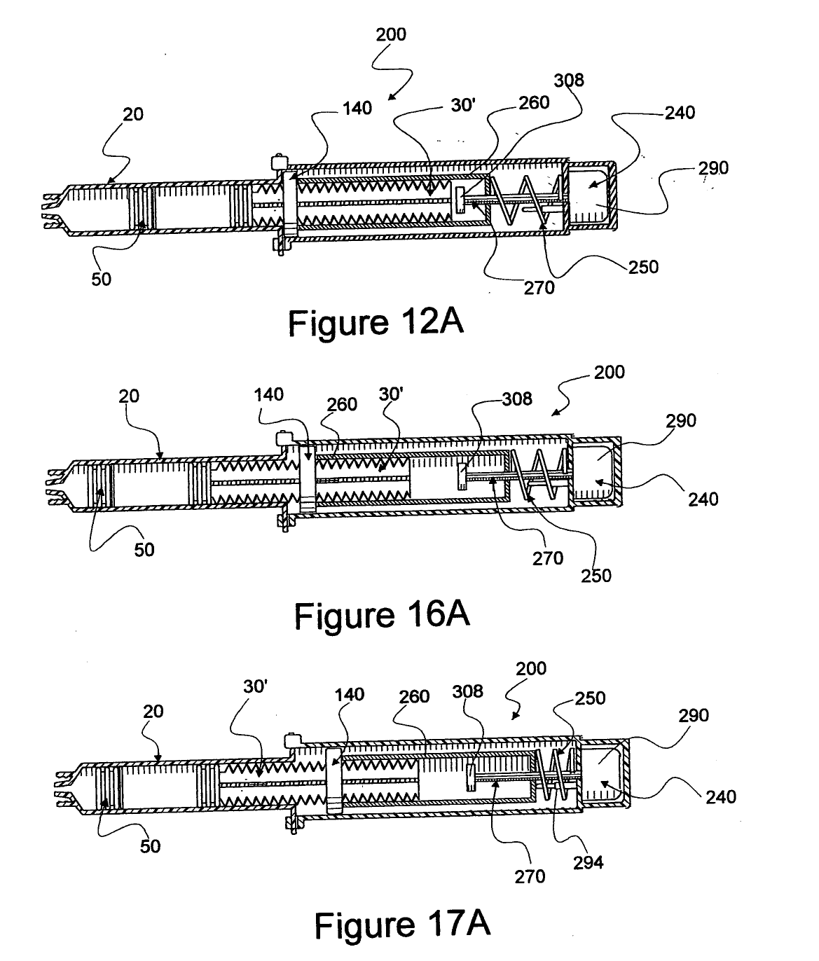

Note, in FIGS. 12 and 15, that drive shaft 270 is a non-circular (hexagon) shaped rod which on one end 302 is slideably displaced through a hole 304 in a proximal face 306 of cylinder 260. Relative to shaft 270, hole 304 is sized and shaped such that when shaft 270 is rotated, cylinder 260 is forced to rotate, but cylinder 260 is freely displaced linearly along the longitudinal axis of shaft 270. As seen in FIG. 11, shaft 270 is securely affixed to a rotor portion of motor 290. As best seen in FIGS. 13, 12A, 16A and 17A, shaft 270 is distally terminated by a stop 308 disposed and securely affixed to shaft 270 within cylinder 260. Stop 308 acts to retain cylinder 260 upon shaft 270 when driver 200 is not connected to a syringe 10′, as seen in FIG. 13.

To connect driver 200 to syringe 10′ (and to collet-button 140), collet-button 140 is displaced to a site near or abutting end 22 (see FIG. 6A). Cylinder 260 is displaced about stem assembly 30′ such that fingers 300 (see FIG. 13) fit into slots 164 (see FIG. 5) as seen in FIGS. 12 and 12A. Note that the state of spring 250 is compressed in FIGS. 12 and 12A when compared to the state of spring 250 in FIG. 13. As compressed in FIGS. 12 and 12A, spring 250 exerts the lower range, e.g. a two pound, force, previously disclosed, upon collet-button 140. Driver 200 is then securely, but releasibly affixed to syringe 10′ as disclosed supra.

To operate driver 200, flow rate is set to a desired, predetermined value by switching the power switch 216 to the “on” state followed by depressing switches 212 and 214 until the desired flow rate is displayed on rate indicator 210. (See FIG. 8.) Rate indicator is preferably a liquid crystal display. Once an appropriate flow rate is set (and all other medical connections are verified), start switch 218 is switch to “on” to initiate driving of the attached syringe.

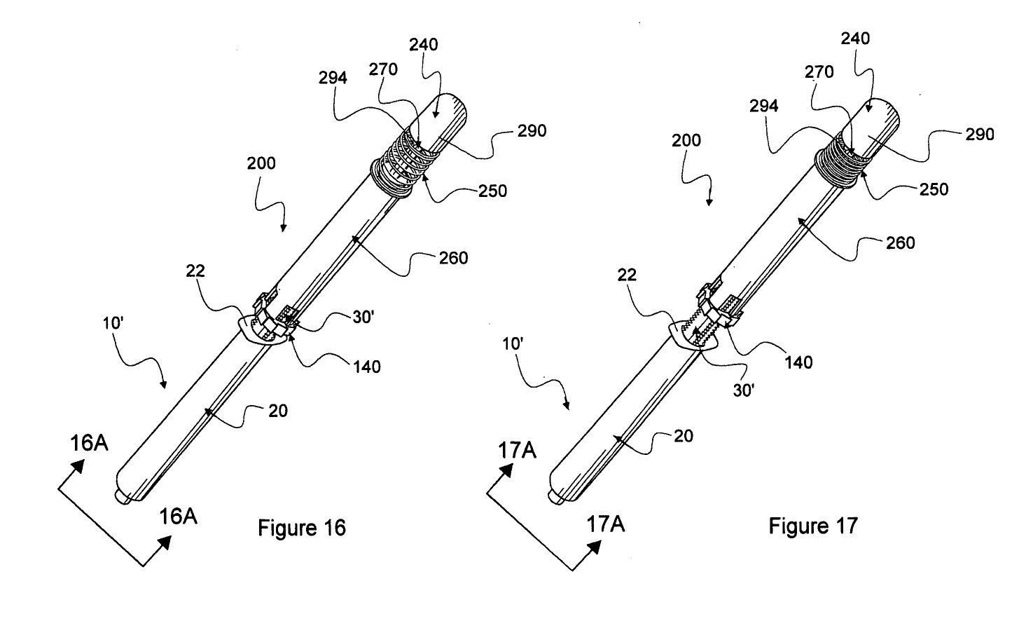

Motor 290 is preferably periodically driven through short increments of time as disclosed in detail hereafter. It is important to note that, to deliver fluid from syringe 10′, motor 290 is powered to rotate shaft 270, cylinder 260 and, therefore, collet-button 140 to selectively rotate collet-button 140, along teeth pattern 170 (or 172) of teeth 110, away from, a first state where collet-button abuts end 22 (see FIGS. 12 and 12A) to a second state where collet-button 140 is displaced proximally from end 22 (see FIGS. 16 and 16A). Such displacement thrusts cylinder 260 proximally thereby compressing spring 250. Responsively, spring 250 forces cylinder 260 linearly distally (a cam action) to force collet-button 140 and associated syringe assembly 30′ to expel fluid from syringe 10′.

As may be noted in FIGS. 17 and 17A, if cylinder 260 is resultingly displaced sufficiently far proximally, contact is made between cylinder 260 and sensor 294. Sensor 294 may be a digital switch, which, when activated, provides an indication of such extreme displacement of cylinder 260. Cause of such displacement is an indication of either to low an effluent flow state from syringe 10′ or an emptying of syringe 10′. In either case, it is advisable to service driver 200 and, therefor, an alarm is generated. (More detail concerning alarms is provided hereafter.) Further, variation of displacement of cylinder 260 as a result of regular rotation of motor 290 may be an indication of a change in displacement force required in an intermediate step. Such a step may be the activation of a chamber dividing valve, such as a valve assembly 50 (see FIG. 1). Sensing such variation (by a sensor which is not shown) would provide an opportunity to vary rates at which fluid flow is driven from a distal chamber 70 when compared to fluid flow driven from a proximal chamber 60.

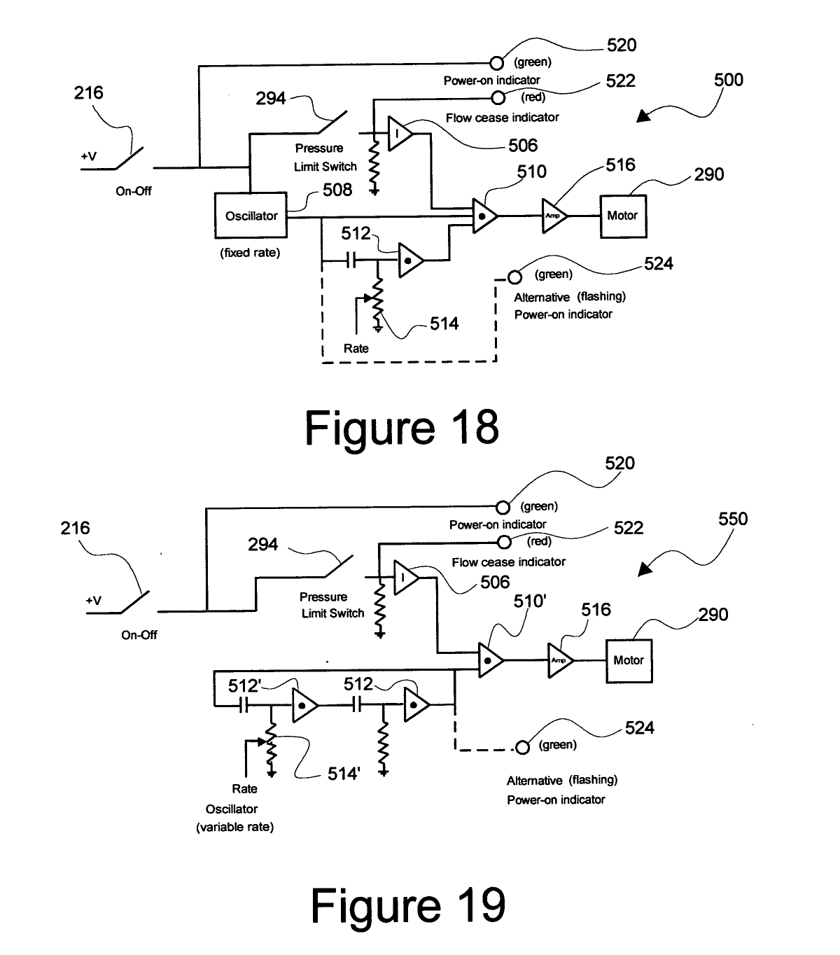

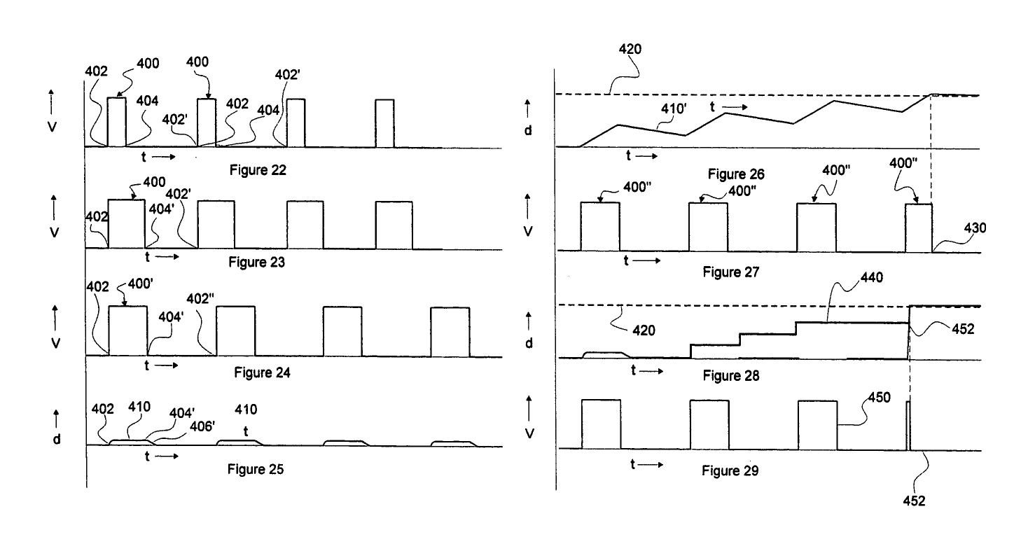

Attention is drawn to FIGS. 18-30 wherein schematics of circuits, waveforms and flow charts depict various modes of control of driver 200. Motor 290 may be variably driven to adjust syringe 10′ effluent fluid flow rates by varying the motor drive voltage. However, it is preferred to adjust syringe effluent flow rates by providing a constant drive voltage for a predetermined period of time at an also predetermined cyclic rate. Reference is now made to FIGS. 22-24 wherein pulse diagrams of motor 290 drive voltage as a function of time is seen. As seen in FIG. 22, motor 290 is driven by a drive pulse 400 through a time (t) beginning at point 402 and ending at point 404. Drive voltage for motor 290 is then removed through a period of time t until the cycle begins again at point 402′. Note that point 402′ becomes point 402 for the next motor 290 drive cycle.

Adjustment of effluent fluid flow rate may be made by adjusting the period between starting and stopping motor 290 wherein time t from point 402 to 402′ is held constant, but drive time is altered from point 402 to a different point 404′, as seen in FIG. 23. Adjustment may also be made by varying the length of the cyclic period as seen when the period seen in FIG. 23 is compared to the period seen in FIG. 24. Note, the time of the drive period in FIG. 23 between points 402 and 404′, is the same as the period between points 402 and 404′ in FIG. 24, but the period between point 404′ and 402′ in FIG. 23 is shorter than the period between 404′ and 402″ in FIG. 24.

Through the drive period (e.g. from 402 to point 404′) drive cylinder 260 is rotated to arcuately displace collet-button 140 about stem assembly 30′ which causes collet-button 140 to be displaced proximally thereby compressing spring 250 via resulting displacement of cylinder 260. An exemplary displacement “d” (pulse 410) of collet-button 140 (and cylinder 260 and spring 250 compression) is plotted in FIG. 25. Note that displacement “d” begins at point 402 and continues until point 404′ when drive upon motor 290 ceases. After a period (until point 406′) decompression of spring 250 respondingly drives cylinder 260 and collet-button 140 distally until collet-button 140 again abuts end 22 (see FIGS. 16 and 16A).

However, should effluent fluid flow from syringe 10′ not clear at a rate commensurate with effluent drive rate, displacement of collet-button 140 (and cylinder 260 and spring 250) may not return to abut collet-button 140 against end 22. In such a case, displacement of collet-button 140 (and cylinder 260 and spring 250) may be continuously displaced, as seen by example by displacement plot 410′ in FIG. 26. In such a case, note that force of spring 250 increases to increase pressure upon fluid in syringe 10′ as force=kx for spring 250. Where “k” is defined to be the constant for spring 250 and “x” is the total compressed distance of spring 250. Thus, as spring 250 is more greatly compressed, force (with resulting pressure) is placed in increasing amounts upon syringe assembly 30′ to expel fluid therefrom. If a restriction to flow continues in spite of the increased force applied by spring 250, displacement plot 410′ may be displaced to a threshold 420. At such a point, it is preferred that power be removed from motor 290 and an alert initiated. Note particularly, that the maximum force (and resulting pressure) which may be imposed upon fluid in syringe 10′ is limited by the compressive force inherent in spring 250 when most compressed, or when collet-button 140 is displaced to threshold 420. Note also that wave form (see FIG. 27) is the same as wave form 400′ (see FIG. 24) to the time 430 where power is removed from motor 290.

In like manner, when syringe 10′ is fully emptied and stem assembly 30′ is fully displaced into barrel 20, collet-button 140 (and cylinder 260 and spring 250) resultingly are also displaced toward threshold 420 as plot of waveform 440 in FIG. 28 exemplifies. In a manner similar to waveform 410′ reaching threshold 420, power is removed at time 452 as displacement reaches threshold 420 as seen in FIG. 29.

A simple control system 500 for regulating driver flow as depicted in FIGS. 22 and 23 is seen in FIG. 18. System 500 comprises a power control switch 216, a displacement limit sensor switch 294, a logic inverter 506, an oscillator 508, an AND gate 510, a variable period one-shot 512 with a rheostat 514 for varying the period of one-shot 512, a motor drive amplifier 516, which drives motor 290, and three status indicators 520, 522 and 524.

Period of oscillation of oscillator 508 determines period from point 402 to 402′ (see FIGS. 22 and 23) of each driver cycle. Power is applied to system 500 by closing switch 216. If switch 294 is not closed, i.e. a threshold displacement of collet-button 140 (and cylinder 260 and spring 250) has not been reached, gate 510 is open to permit motor 290 to be driven. At a predetermined point in each cycle of oscillator 508, one-shot 512 fires for a period which is determined by a setting of rheostat 514 to drive motor 290. Thus, once per oscillator 508 cycle, motor 290 is driven as depicted in FIGS. 22 and 23. A different rate is determined by adjusting rheostat 514. A “power on” indication is provided by either indicator 520 or 524. Note, that indicator 520 provides a constant illumination when switch 216 is on while indicator 524 provides a flashing indicator, displaying oscillatory rate. When switch 294 is closed, indicator 522 displays an alert state and inverter 506 closes gate 510 removing power from motor 290 through amplifier 516. Also note, that if switch 294 is opened through dynamics of spring 250, indicator 522 is extinguished and normal motor 290 operation resumes.

A control circuit 550 for variable period driver 200 cycle is seen in FIG. 19. Circuit 550 varies pump rate by adjusting driver cycle period as differentiated between time between points 402 and 402′ in FIGS. 23 and 402 and 402″ in FIG. 24. As seen in FIG. 19, control circuit 550 comprises a power control switch 216, a displacement limit sensor switch 294, a logic inverter 506, an AND gate 510′, a variable period one-shot 512′ with a rheostat 514′ for varying the period of one-shot 512′ and a linked one-shot 512 connected with one-shot 512′ to provide a variable period oscillator, a motor drive amplifier 516, which drives motor 290, and three status indicators 520, 522 and 524.

Period of oscillation of oscillator 508 determines period from point 402 to 402′ (see FIGS. 23) and from point 402 to 402″ (see FIG. 24) of each driver cycle, based upon varied settings of rheostat 514′. Power is applied to system 500 by closing switch 216. If switch 294 is not closed, i.e. a threshold displacement of collet-button 140 (and cylinder 260 and spring 250) has not been reached, gate 510′ is open to permit motor 290 to be driven. At a predetermined point in each cycle, one-shot 512 fires for a fixed period during an overall period which is determined by a setting of rheostat 514′ to drive motor 290. Thus, once per cycle, motor 290 is driven as depicted in FIGS. 23 and 24. A different oscillator rate is determined by adjusting rheostat 514′. A “power on” indication is provided by either indicator 520 or 524. Note, that indicator 520 provides a constant illumination when switch 216 is on while indicator 524 provides a flashing indicator, displaying oscillatory rate. When switch 294 is closed, indicator 522 displays an alert state and inverter 506 closes gate 510 removing power from motor 290 through amplifier 516. Also note, that if switch 294 is opened through dynamics of spring 250, indicator 522 is extinguished and normal motor 290 operation resumes.

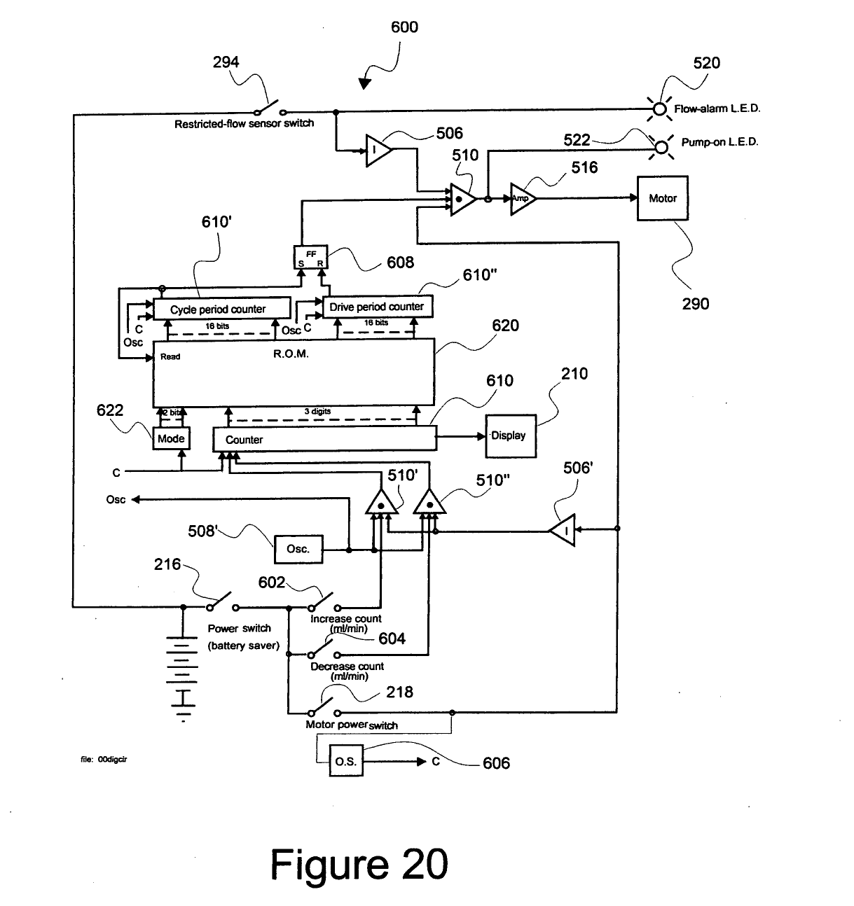

A significant requirement of driver 200 operation may be a requirement to control effluent flow rate of a wide range of values. As an example, it may be desirable to vary flow over a predetermined range from 0.1 ml/hour to 100 ml/hour. Precisely setting and achieving such a range is difficult using rheostatic control. For this reason, a digital control system such as system 600, seen in FIG. 20, may be preferred. As seen in FIG. 20, a digital control system 600 may comprise a power control switch 216, a displacement limit sensor switch 294, a rate incrementing switch 602, a flow rate decrementing switch 604, an oscillator 508′, two logic inverters 506 and 506′, three AND gates (each numbered 510, 510′ and 510″), a motor power switch 218, a power-on one shot 606, a flip-flop 608, three counters (each numbered 610, 610′ and 610″), a read only memory 620, a mode control register 622, an operating status display 210 (see FIG. 9), a motor drive amplifier 516, which drives motor 290, and two status indicators 520 and 522.

Operation of control system to that disclosed supra for control systems 500 and 550, with some notable exceptions. Flow rates are incremented and decremented by depressing switches 602 and 604, respectively, to adjust desired flow rate which is stored in counter 610 and visually fed-back via display 630 (see FIG. 9). Oscillator 508′ is used to gate inputs (to AND gates 510 and 510′) from inputs from switches 602 and 604 to limit rate of change of display 630. Further, closing of power switch 216 to an “on” state inhibits input by switches 602 and 604 through gates 510 and 510′, respectively, to deter changing rate while motor 290 is being driven. Counter 610 is preferably a base-ten counter for easier interpretation by display 630.

Counter 610, in conjunction with mode register 622, comprises an addressing register for read only memory 620. Memory 620 may hold a unique drive period and total cycle period for each setting of counter 610, thereby permitting predetermination of optimum drive to null (no motor drive) periods for various flow rate settings. Note that counter 610 may be adjusted until switch 218 is closed. At such time, initial conditions are generated by output of one shot 606 which clears counters 610′ and 610″ and sets mode 622 to a desired operating mode (e.g. 0, for normal operating mode). Operating modes may be changed to change flow rates based upon predetermined conditions, such as detecting emptying of a chamber 70 to drive effluent from chamber 60 (see FIG. 2) at a different rate. Procedures for changing mode register 622 are not addressed further herein.

Once desired flow rates are set and syringe 10′ is affixed to driver 200 and ready for fluid delivery, switch 218 is closed to initiate driver 200 operation. Each counter 610′ and 610″ counts down to underflow which yields a “borrow signal” from the least significant bit of each counter (or like signal). Underflow of counter 610 sets flip-flop 608 to initiate a motor 290 drive period through gate 510 and amplifier 516. When counter 610″ counts down to underflow, flip-flop 608 is reset to terminate the current motor 290 drive period. The cycle period determining number set in counter 610′ is greater than the motor 290 drive period number set in counter 610″ which makes the cycle period longer than the motor 290 drive period.

Note that status indicator 522 is turned “on” when switch 294 is closed, to indicate an alarm condition, generally for the same reasons the same alarm indicator 522 in FIGS. 18 and 19 is illuminated. In the case of status indicator 522, the motor 290 on signal is turned “on” each cycle motor 290 is being driven, providing a flashing indicator of driver operation.

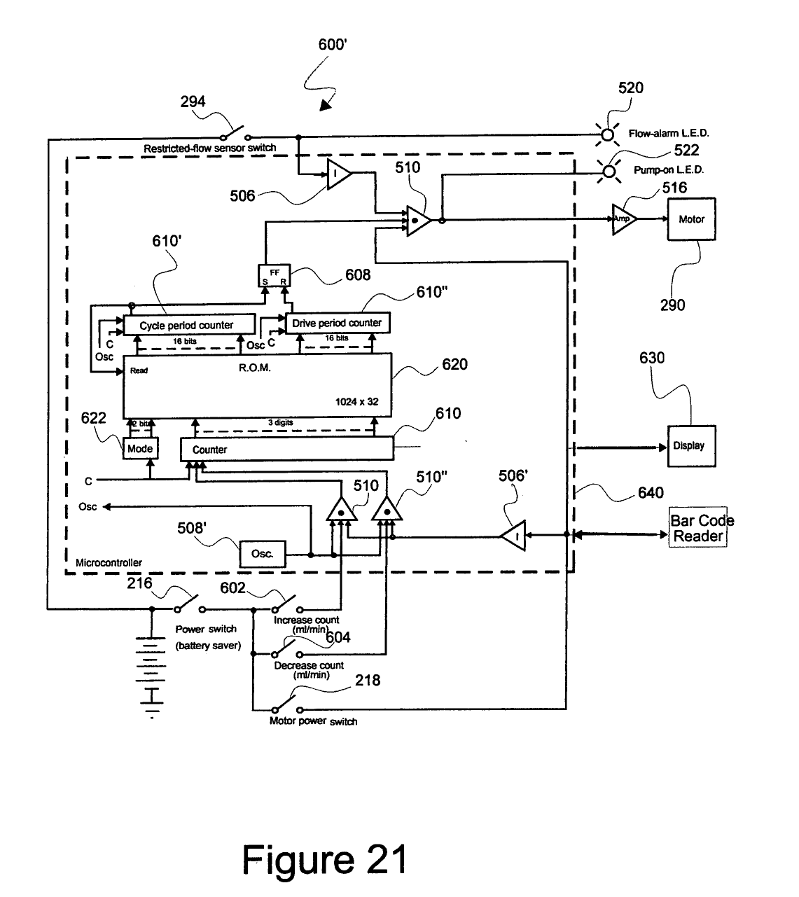

A microprocessor based control system 600′ is seen in FIG. 21. Note that no initial conditioning one shot 606 is required in the logic diagram of FIG. 21 (compared to the logic seen in FIG. 20) and that a microprocessor identified by dashed line 640 replaces individual components enclosed within dashed line 640. A bar code reader 642 is added to system 600′. Interfaces and programs for microprocessor and bar code readers are well in the digital computer art.

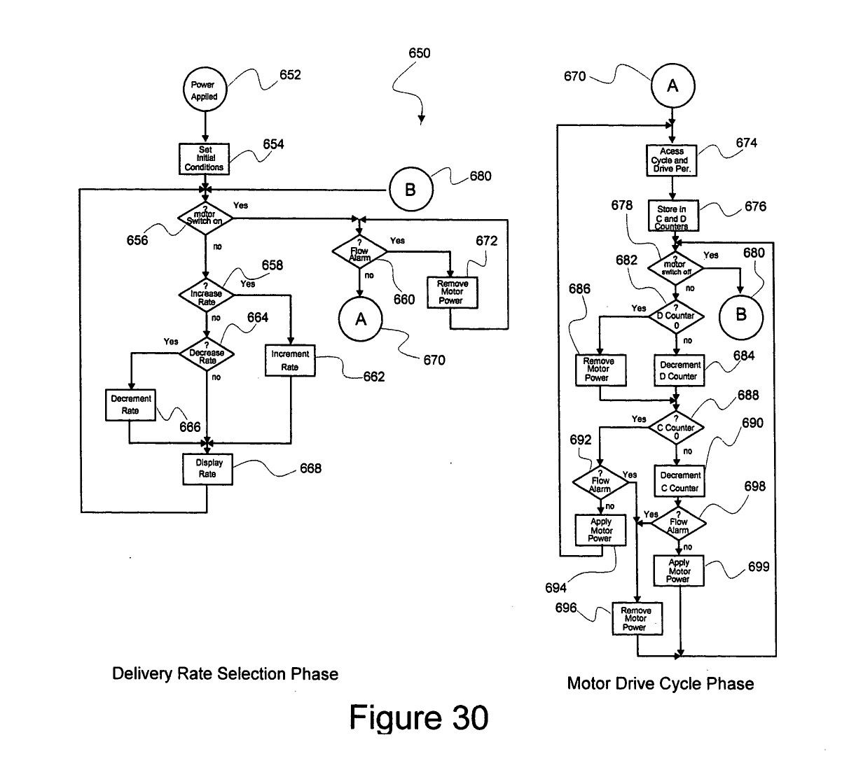

A program flow diagram 650 for operation of driver 200 under control of system 600′ is seen in FIG. 30. In general, circles, such as circle 652, are program initiating or flow connecting points. Rectangles represent computational and control functions. Diamonds represent decision functions.

Program entry 652 begins with closing of switch 216, see FIG. 21. Function 654 sets initial conditions for all flags and registers, such process are well understood and programmed for microprocessor initialization procedures. At decision 656, a flow path choice is made to proceed to decision 658 if switch 218 is open otherwise flow proceeds to decision 660. At decision 658, a flow path choice is made to proceed to increment the contemporarily displayed desired flow rate (function 662) if switch 602 is closed or, otherwise to proceed to decision 664. At decision 664 a choice is made to decrement the contemporarily displayed desired flow rate (function 666) if switch 604 is closed or to proceed to function 668 to display the current flow rate. Note that program path from functions 662 and 666 also proceed to function 668. Program flow from function 668 reenters decision 656.

If program flow proceeds to decision 660, a test is made to see if an alarm flag is set. If so, another flag is set to remove power from motor 290 (see FIGS. 26 and 28) via function 672 and continues flow to decision 660. If not, program flow continues to connecting bubble 670.

From connection 670, program flow is designed to control total cycle and motor drive periods, beginning at function 674. Function 674 accesses total cycle period count and motor drive period count from counter rate determined and displayed in function 668. Function 676 follows function 674 and loads new cycle and motor drive counts into associated “c” (cycle period) and “d” (drive period) registers (or memory cells), respectively. Flow then proceeds to decision 678 whereat a choice is made to proceed to connection if switch 218 (see FIG. 21) is open or to proceed to decision 682 if switch 218 is closed.

At decision 682, a choice is made to proceed to function 684 if contents of the “d” register is not zero or to proceed to function 686 if contents of the “d” register is zero. Function 684 decrements contents of the “d” register. Function 686 sets a flag to remove motor power. From both functions 684 and 686, program flow continues to decision 688.

At decision 688, a choice is made to proceed to proceed to function 690 if contents of the “c” register are not zero or, otherwise, to proceed to decision 692. Function 690 decrements contents of the “c” register or memory cell. At decision 692 a choice is made to proceed to function 694 if switch 294 (see FIG. 21) is open or to proceed to function 696 if switch 294 is closed, indicating an alarm condition, precursors of which are disclosed supra. At function 696, a motor power “on” flag is reset to remove power from motor 290 and an alarm flag is set. Program flow then proceeds to decision 678. At function 694, any alarm flag is reset and a motor power “on” flag is set to assure continuance of motor 290 operation. Program flow then proceeds to function 674.

From function 690, flow continues to decision 698 wherein a choice is made to proceed to function 696 if switch 294 is closed or to proceed to otherwise proceed to function 699. Flow from function 696 is as disclosed supra. Via function 699, the motor “on” flag is set to assure motor 290 will be on if not reset by decision 682. Program flow from continuation 680 returns to decision 656.

Use of a spring, such as spring 250 (see FIGS. 11, 12, 14, 16 and 17) to limit maximum pressure in a syringe, as disclosed supra, permits use of syringe 10′ and, especially, collet-button 140 in a manner which provides flow rate curtailing safety in a manual syringe driver. Reference is now made to FIG. 31 wherein a manual driver 700 is seen to be affixed to a syringe 10′. Note that stem assembly 30′ has been removed from collet-button 140 and syringe 10′ so that parts of driver 700 may be more clearly visualized. Even so, it is necessary to have stem assembly 30′ affixed to syringe 10′ and collet-button 140 (as seen in FIG. 6A) for driver 700 to operate.

Manual driver 700 comprises a spring 250 and a housing 710 which acts as a “lock apparatus” which houses spring 250 and is releasibly affixed to syringe 10′. Similar to housing 190 (see FIG. 10) housing 710 is facilely, but securely affixed to syringe 10′ by a bayonet attachment 712 about extensions 45 and 45′ (see FIG. 10) of syringe 10′. As seen in FIG. 31, housing 710 comprises a cupped part 714 having opposing latching edges 716 and 718 which fit about syringe 10′ lateral extension 45 to be caught thereat due to a compressed force in spring 250 when driver 700 is so disposed. On the side opposing part 714, housing 710 has a similar bayonet attachment, assigned number 712′, but mostly hidden in FIG. 31.

Further, housing 710 comprises a pair of risers 720 and 722 which extend superiorly from attachments 712 and 712′ to be joined by a hollow ringed connection 724 at the top thereof. Connection has an orifice 726 which is sufficiently large to permit a stem, such as stem 80 (see FIG. 3A), and stem end 130 (see FIG. 6A) to pass therethrough. With collet-button 140 disposed as seen in FIG. 31, a set of vertical notches 152 form a ratchet-like surface 730. A user may interact digitally with surface 730 to articulate collet-button 140 to along a pattern of teeth 120 (see FIG. 3). Thereby, collet-button 140 is displaced away from syringe 10′ and spring 250 is simultaneously compressed. At least one raised surface 740 is disposed on an inner surface 742 of riser 722 to form a pawl against one direction (see arrow 744) of rotation of collet-button 140. Thus collet-button 140 can only be rotated in one direction (see arrow 746) to compress spring 250. A compressed spring 250 responds to force collet-button 140 linearly toward syringe 10′, thereby providing a cam action which translates rotary motion of collet-button 140 to linear displacement of collet-button 140 to thereby restrict force applied to collet-button 140 and stem 80 to force of energy stored in spring 250. Note that, by pulling upon an associated stem assembly 30′, spring 250 may be compressed as a volume of fluid is drawn into syringe 10′, for such purposes as checking quality of needle insertion through blood flash. Once such a check is complete, letting loose of stem assembly 30′ permits energy so stored in spring 250 to automatically return stem assembly 30′ to a pre-check state.

The inventions disclosed herein may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed and desired to be secured by Letters Patent is:

1. A method for powered dispensing of fluid from a medical syringe comprising the steps of:
(a) selecting a syringe having a partially closed distal end through which fluid is dispensed, an open proximal end and a cylindrical barrel therebetween, said barrel further comprising lateral extensions which extend radially outward at the proximal end;
(b) providing a plunger piston for said syringe, said piston comprising a plunger on the end to be distally disposed which is sized and shaped to compressibly occlude the barrel when disposed therein, a collet-button on the end to be proximally disposed and a stem therebetween which comprises a shaft which further comprises a plurality of spirally oriented grooves along the length thereof, the collet-button having a hollow core with an internal surface having annular a spiral thread disposed therein, the thread of which cooperatively operates with grooves disposed on the shaft in a screw-nut relationship, and a proximally facing structure for interfacing with a radially impelled driver component;
(c) providing a syringe driver comprising:
(i) a driver housing which is securely affixable to said barrel;
(ii) a motor disposed within said housing to be in line with said barrel when affixed to said housing, said motor having sufficient torque to displace the piston to dispense fluid from said syringe;
(iii) a drive shaft for rotationally displacing a driver component affixed to said motor;
(iv) the driver component comprising a driver part and drive shaft interface to the drive shaft by which drive torque from said motor only rotationally actuates said driver part, said driver part comprising a connecting interface which couples to said structure to said collet-button when in contact therewith to transfer torque from said motor therethrough;
(v) an energy storage device disposed between said motor and said driver part such that displacement of the driver part toward said motor stores energy in the energy storage device;
(vi) a source of power for said motor;
(vii) on-off switch control for connecting and disconnecting the power source to and from said motor, respectively;
(viii) a sensor which determines when displacement of the driver part toward the motor reaches a predetermined site and a predetermined amount of energy is, therefore, stored in said energy storage device;
(d) displacing said plunger piston into said syringe;
(e) introducing fluid into said syringe barrel;
(f) affixing the syringe driver to the syringe such that the connecting interface of the driver component engages the collet-button structure;
(g) applying power to the motor via the on-off control;
(h) thereby, rotating and linearly displacing said collet-button to store energy in said energy storage device;
(i) displacing the plunger piston distally by force of release of energy from the energy storage device to thereby dispense fluid from the syringe.

2. The method for powered dispensing of fluid from a medical syringe according to claim 1 wherein the providing sensor step comprises providing a power-off sensor switch which is activated by said sensor.

3. The method for powered dispensing of fluid from a medical syringe according to claim 2 further comprising a step of sensing that displacement of the driver part has reached the predetermined site and activating said sensor switch to remove power from said motor thereby limiting amount of force which may be applied to pressurize fluid distally disposed from said plunger.

4. The method for powered dispensing of fluid from a medical syringe according to claim 1 wherein the on-off switch control providing step comprises providing an on-off switch.

5. The method for powered dispensing of fluid from a medical syringe according to claim 4 comprising a further step of turning the on-off switch to off.

6. The method for powered dispensing of fluid from a medical syringe according to claim 1 wherein the affixing step transfers energy into the energy storage device and thereby inhibits reflux of fluid proximally toward said syringe when power is removed from said motor.

7. The method for powered dispensing of fluid from a medical syringe according to claim 1 wherein said piston providing step comprises providing a heat stake which releasibly affixes the collet-button to the proximal end of the stem whereby the collet-button provides a gripping surface which may be used when the syringe is unattached to the syringe driver.

8. The method for powered dispensing of fluid from a medical syringe according to claim 1 wherein the affixing step comprises a further step of fracturing the heat stake whereby the collet-button is freed for displacement along the shaft of the grooved stem.

9. The method for powered dispensing of fluid from a medical syringe according to claim 8 wherein the affixing step comprises yet a further step of displacing the collet-button along the shaft of the grooved stem until the collet-button is in contact with the proximal end of said barrel.

10. The method for powered dispensing of fluid from a medical syringe according to claim 1 wherein said driver housing providing step comprises providing a bayonet attachment by which the driver housing is facilely, yet securely, releasibly affixed to the lateral extensions of said syringe.

11. The method for powered dispensing of fluid from a medical syringe according to claim 10 wherein said affixing step comprises applying compressive pressure to said driver part and said energy storage device to thereby securely affix said bayonet attachment to the lateral extensions of the syringe.

12. The method for powered dispensing of fluid from a medical syringe according to claim 1 wherein said motor providing step comprises providing an electric motor.

13. The method for powered dispensing of fluid from a medical syringe according to claim 1 wherein said on-off control providing step comprises providing circuitry for intermittently driving said motor.

14. The method for powered dispensing of fluid from a medical syringe according to claim 13 wherein said circuitry providing step comprises providing circuitry for selecting a driving period which determines a rate at which fluid is dispensed from said syringe.

15. The method for powered dispensing of fluid from a medical syringe according to claim 13 wherein said circuitry providing step comprises providing a microprocessor based controller.

16. The method for powered dispensing of fluid from a medical syringe according to claim 15 comprising further steps associated with metering and processing dynamics of pressure feedback associated with energy stored within the energy storage device to enhance operation of the syringe driver.

17. The method for powered dispensing of fluid from a medical syringe according to claim 16 wherein said syringe providing step comprises providing a syringe having at least one chamber separating stopper disposed in the barrel thereof to thereby provide a multi-chamber syringe.

18. The method for powered dispensing of fluid from a medical syringe according to claim 17 wherein said metering and processing steps comprise differentiating flow rates between sequential deliveries of disparate fluids disposed within the multi-chamber syringe.

19. The method for powered dispensing of fluid from a medical syringe according to claim 15 wherein said microprocessor based controller providing step comprises providing a bar code reader.

20. The method for powered dispensing of fluid from a medical syringe according to claim 19 comprising a further step of using said bar code reader to read patient and drug identification for the purpose of imposing features such as programmable drug data bases with automatic lock-out, alerts and alarms restrictions on syringe driver use.

21. The method for powered dispensing of fluid from a medical syringe according to claim 1 comprising the step of intermittently driving the driver part with a torque from the motor which would yield an over-pressure force to a piston if driven directly to a plunger piston, but which provides a reduced and smoothed reasonably acceptable pressure to the plunger through the energy storage device by actuating the driver part to intermittently drive against the energy storage device and therethrough to the plunger piston.

22. A syringe driver system comprising:
a syringe comprising a hollow cylindrical barrel which is open at a proximal end and partially closed at a distal end through which fluid flows, said barrel further comprising extensions which outwardly extend laterally at the proximal end thereof;
a piston comprising a plunger which is compressibly and occludably disposed in said barrel and which is thereby used to displace fluid within the barrel;
said piston further comprising stem apparatus which is securely affixed to said plunger and which extends proximally therefrom;
said stem apparatus comprising an elongated stem, having a plurality of external spirally oriented grooves disposed along the length thereof, and a collet-button releasibly affixable to the proximal end of the stem;
said collet-button comprising a hollow core, said core comprising an internal, nut-like spiral screw thread which is sized and pitched for facile, angular displacement along the grooves of said stem to linearly displace the collet-button relative to the stem;
said collet-button further comprising a proximally facing structure for interfacing with a radially impelled driver component;
a syringe driver comprising:
a driver housing which is securely affixable to said barrel;
a motor disposed within said housing in line with said barrel when affixed to said housing, said motor having sufficient torque to displace the piston to dispense fluid from said syringe;
a drive shaft for only rotationally displacing a driver component affixed to said motor;
the driver component comprising a driver part and drive shaft interface to the drive shaft by which drive torque from said motor rotationally displaces said driver part, said driver part comprising a connecting interface which couples to said collet-button facing structure to transfer torque from said motor to said collet-button when in contact therewith;
an energy storage device disposed between said motor and said driver part such that displacement of the driver part toward said motor stores energy in the energy storage device;
a source of power for said motor;
on-off control for connecting and disconnecting power from said motor;
a sensor which determines when displacement of the driver part toward the motor has reached a predetermined site and a predetermined amount of energy has been stored in said energy storage device.

23. The syringe driver system according to claim 22 further comprising a power-off sensor switch which is activated by said sensor when the driver part has reached the predetermined site to remove power from said motor to thereby limit the amount of force which can be applied to pressurize fluid distally disposed from said plunger.

24. The syringe driver system according to claim 22 wherein the on-off switch control comprises an on-off switch by which syringe driver system operation is manually terminated.

25. The syringe driver system according to claim 22 wherein said piston further comprises a heat stake which releasibly affixes the collet-button to the proximal end of the stem whereat the collet-button may be facilely gripped when the syringe is unattached to the syringe driver and which is fractured to free the collet-button to be displaced along the shaft of the grooved stem.

26. The syringe driver system according to claim 22 wherein said driver housing comprises a bayonet attachment by which the driver housing is facilely, yet securely, and releasibly affixed to said syringe.

27. The syringe driver system according to claim 22 wherein said motor is an electric motor.

28. The syringe driver system according to claim 22 wherein said on-off control comprises electrical circuitry for intermittently driving said motor.

29. The syringe driver system according to claim 28 wherein said electrical circuitry comprises apparatus for selecting a rate at which fluid is dispensed from said syringe.

30. The syringe driver system according to claim 28 wherein said electrical circuitry comprises a microprocessor based controller.

31. The syringe driver system according to claim 30 wherein said microprocessor based controller comprises a bar code reader.

32. A driver system for a medical syringe comprising:
a housing which comprises parts whereby the housing is securely, but releasibly affixed to the medical syringe;
a drive motor disposed within the housing in line with the long axis of the syringe, said drive motor comprising sufficient torque to displace a stem of the medical syringe to thereby dispense fluid from the syringe;
a power source for said motor;
a cam interface between the motor and the syringe stem, said interface disposed to transform rotational displacement of the motor to a linear displacement in a direction opposite to direction of fluid flow from the syringe;
an energy storage device disposed to store energy received from the linear displacement and, thereby, provide an oppositely directed force to responsively dispense fluid from the syringe, said energy storage device having limited energy storage and energy responsive capacity to thereby limit the force which is ultimately provided to dispense the fluid from the syringe.

33. The driver system for a medical syringe according to claim 32 further comprising a manual control system for stopping and starting dispensing of fluid and for controlling dispensing rate.

34. The driver system for a medical syringe according to claim 33 wherein said manual control system comprises an on-off switch.

35. The driver system for a medical syringe according to claim 33 wherein said manual control system comprises rate control setting apparatus.

36. The driver system for a medical syringe according to claim 35 further comprising rate control apparatus for controlling fluid dispensing rate.

37. The driver system for a medical syringe according to claim 36 wherein said rate control apparatus comprises an intermittent drive system having a first predetermined period during which the motor is turned “on” and a second predetermined period during which the motor is turned “off”, the frequency and length of time of each period determining the dispensing fluid flow rate.

38. The driver system for a medical syringe according to claim 32 wherein said drive motor is an electric motor.

39. The driver system for a medical syringe according to claim 32 further comprising a medical syringe comprising a hollow cylindrical barrel which is open at a proximal end and partially closed at a distal end through which fluid flows;
a piston comprising a plunger which is compressibly and occludably disposed in said barrel and which is thereby used to displace fluid within the barrel;
said piston further comprising stem apparatus which is securely affixed to said plunger and which extends proximally therefrom;
said stem apparatus comprising an elongated stem, having a plurality of external spirally oriented grooves disposed along the length thereof, and a collet-button releasibly affixable to the proximal end of the stem;
said collet-button comprising a hollow core, said core comprising an internal, nut-like screw thread which is sized and pitched for facile, angular displacement along the grooves of said stem to linearly displace the collet-button relative to the stem.

40. The driver system for a medical syringe according to claim 32 wherein the cam interface comprises a drive apparatus which transfers rotational energy from the motor to the collet-button to resultingly displace the collet-button about the grooves of the stem and thereby linearly along the stem.

41. A syringe driver system comprising:
a syringe comprising a hollow cylindrical barrel which is open at a proximal end and partially closed at a distal end through which fluid flows;
a piston comprising a plunger which is compressibly and occludably disposed in said barrel and which is thereby used to displace fluid within the barrel;
said piston further comprising stem apparatus which is securely affixed to said plunger and which extends proximally therefrom;
said stem apparatus comprising an elongated stem, having a plurality of spirally oriented grooves disposed along the length thereof, and a collet-button releasibly affixable to the proximal end of the stem;
said collet-button comprising a hollow core, said core comprising an internal, nut-like screw thread which is sized and pitched for facile, angular displacement along the grooves of said stem to linearly displace the collet-button relative to the stem.

42. The syringe driver system according to claim 41 further comprising a heat stake disposed to releasibly affix said collet-button to the proximal end of said stem.

43. The syringe driver system according to claim 41 wherein said barrel further comprises extensions which outwardly extend laterally at the proximal end thereof.

44. The syringe driver system according to claim 41 further comprising snap-on lock apparatus whereby the collet-ton, button, when disposed at the proximal end of the barrel, is securely affixed by the apparatus to retard linear displacement of the collet-button while permitting rotation thereof to propel the stem linearly.

45. The syringe driver system according to claim 44 wherein said lock apparatus comprises at least one direction retarding pawl and said collet-button comprises corresponding ratchet teeth which restrict collet-button rotation to a single direction.

46. The syringe driver system according to claim 45 wherein said pawl and corresponding ratchet teeth restrict rotation such that the single direction of collet-button rotation displaces the collet-button away from said syringe barrel.

47. The syringe driver system according to claim 46 wherein said lock apparatus comprises a compartment for an energy storage device.

48. The syringe driver system according to claim 47 wherein said lock apparatus further comprises an energy storage device disposed to store energy as the collet-button is displaced away from the syringe barrel.

49. The syringe driver system according to claim 48 wherein said energy storage device comprises an interface within the compartment which resultingly releases stored energy to drive the collet-button and syringe stem in a direction which dispense fluid from the syringe.

50. The syringe driver system according to claim 48 wherein said energy storage device is a spring which is compressed when the collet-button is rotated to be linearly displaced away from the barrel.

51. The syringe driver system according to claim 41 further comprising a housing which comprises parts whereby the housing is securely, but releasibly affixed to the medical syringe;
a drive motor disposed within the housing, said drive motor comprising sufficient torque to displace a stem of the medical syringe to thereby dispense fluid from the syringe;
a power source for said motor;
a cam interface between the motor and the syringe stem, said interface disposed to transform rotational displacement to a linear displacement in a direction opposite direction of displacement necessary to dispense fluid from the syringe;
an energy storage device disposed to store energy resulting from the linear displacement and, thereby, provide an oppositely directed force to responsively dispense fluid from the syringe, said energy storage device having limited energy storage and energy responsive capacity to thereby limit force which is ultimately provided to dispense the fluid from the syringe.
